(19) United States
(12) Patent Application Publication
(10) Pub. No.: US 2021/0259972 A1
(43) Pub. Date: Aug. 26, 2021

(54) THROMBOLYTIC MICROSPHERES TO DISSOLVE VASO-OCCLUSIVE CLOTS

(71) Applicant: Richard C.K. Yen, Yorba Linda, CA (US)

(72) Inventor: Richard C.K. Yen, Yorba Linda, CA (US)

(21) Appl. No.: 17/208,736

(22) Filed: Mar. 22, 2021

Related U.S. Application Data
(63) Continuation-in-part of application No. 15/233,779, filed on Aug. 10, 2016.
Application No. 15/233,779 is a continuation-in-part of application No. 13/560,727, filed on Jul. 27, 2012, now abandoned.
Application No. 13/560,727 is a continuation-in-part of application No. 14/226,544, filed on Mar. 26, 2014, now Pat. No. 9,629,931.
Application No. 14/226,544 is a continuation-in-part of application No. 12/927,543, filed on Nov. 16, 2010, now Pat. No. 9,226,898.
Application No. 12/927,543 is a continuation-in-part of application No. 13/560,727, filed on Jul. 27, 2012, now abandoned.
Application No. 13/560,727 is a continuation-in-part of application No. 13/604,770, filed on Sep. 6, 2012, now Pat. No. 9,351,925.
Application No. 13/604,770 is a continuation-in-part of application No. 13/605,765, filed on Sep. 6, 2012, now Pat. No. 9,504,641.
Application No. 15/233,779 is a continuation-in-part of application No. 14/925,506, filed on Oct. 28, 2015, now abandoned.
Application No. 14/925,506 is a continuation-in-part of application No. 13/560,727, filed on Jul. 27, 2012, now abandoned.
Application No. 13/560,727 is a continuation-in-part of application No. 13/604,770, filed on Sep. 6, 2012, now Pat. No. 9,351,925.
Application No. 14/925,506 is a continuation-in-part of application No. 13/605,765, filed on Sep. 6, 2012, now Pat. No. 9,504,641.
Application No. 13/605,765 is a continuation-in-part of application No. 16/505,257, filed on Jul. 8, 2019.
Application No. 16/505,257 is a continuation-in-part of application No. 15/233,779, filed on Aug. 10, 2016.
Application No. 15/233,779 is a continuation-in-part of application No. 15/618,234, filed on Jun. 9, 2017, now Pat. No. 10,603,287.
Application No. 16/505,257 is a continuation-in-part of application No. 14/953,066, filed on Nov. 27, 2015, now abandoned.
Application No. 14/953,066 is a continuation-in-part of application No. 15/238,928, filed on Aug. 17, 2016, now abandoned.
Application No. 15/238,928 is a continuation-in-part of application No. 17/094,114, filed on Nov. 10, 2020.
Application No. 17/094,114 is a continuation-in-part of application No. 15/233,779, filed on Aug. 10, 2016.
Application No. 15/233,779 is a continuation-in-part of application No. 16/505,257, filed on Jul. 8, 2019.
Application No. 16/505,257 is a continuation-in-part of application No. 14/953,066, filed on Nov. 27, 2015, now abandoned.
(60) Provisional application No. 61/853,041, filed on Mar. 27, 2013.
(60) Provisional application No. 61/573,630, filed on Sep. 10, 2011.
(60) Provisional application No. 61/627,623, filed on Oct. 14, 2011.
(60) Provisional application No. 61/573,630, filed on Sep. 10, 2011.
(60) Provisional application No. 61/627,623, filed on Oct. 14, 2011.
(60) Provisional application No. 62/733,468, filed on Sep. 19, 2018.
(60) Provisional application No. 62/364,764, filed on Jul. 20, 2016.
(60) Provisional application No. 63/100,670, filed on Mar. 25, 2020.

(51) Int. Cl. A61K 9/16; A61K 38/49; A61K 38/36; A61K 9/51; A61K 9/00

(52) U.S. Cl. CPC A61K 9/1658; A61K 38/49; A61K 9/0019; A61K 9/5169; A61K 38/363

(57) ABSTRACT

A product, composition, suspension and/or method to treat subjects infected with respiratory viruses, such as coronavirus, and in relieving hypoxic condition. A suspension of thrombolytic albumin nanospheres that can target vaso-occlusive clots in vivo and then lyse the obstructive blood clots with high efficiency can be intravenously administer to a subject in need thereof. The nanospheres can encapsulate an enzymatically active bioreactive agent, such as urokinase. Since the active enzyme is targeting and binding essentially only to obstructive clots, there is minimal risk of side-effects to the subject. The nanospheres are less than one micron in diameter, resulting in a size much smaller than red blood cells.

### CROSS-REFERENCE TO RELATED APPLICATION

This application claims the benefit of priority under 35 U.S.C. § 119(e) based upon co-pending U.S. provisional patent application Ser. No. 63/100,670 filed on Mar. 25, 2020. The entire disclosure of the prior provisional application is incorporated herein by reference.

This application is a continuation-in-part under 35 U.S.C. § 120 based upon co-pending U.S. patent application Ser. No. 17/094,114 filed on Nov. 10, 2020, co-pending U.S. patent application Ser. No. 16/505,257 filed on Jul. 8, 2019, co-pending U.S. patent application Ser. No. 15/618,234 filed on Jun. 9, 2017, and co-pending U.S. patent application Ser. No. 15/233,779 filed on Aug. 10, 2016, which are incorporated herein by reference in their entirety.

### BACKGROUND

### Technical Field

The present technology involves a product and its method of manufacture which can accelerate the localized dissolving or break-up of harmful clots after its administration to a patient suffering from occlusive clots within the vascular system including the pulmonary circuit, which can cause severe morbidity and mortality, such as heart attack, stroke, pulmonary embolism, and lung damage from infectious agents. The present technology relates to a thrombolytic microspheres for use in connection with dissolving vaso-occlusive clots.

### Background Description

The occurrence of unwanted clots in the vascular system can lead to severe morbidity and mortality depending on where the clot occurs. These vascular clots can be described as vaso-occlusions, or relating to, resulting from, or caused by occlusion of a blood vessel. If the clot occurs in the arteries of the heart, it can cause a heart attack. If the obstruction occurs in the brain, it can lead to a stroke. Some patients will form large clots in their legs which can break off and flow to the lung, causing a pulmonary embolism. Infectious agents such as the flu virus and the coronavirus can damage the endothelium of the blood vessels in the lung resulting in platelet clots which then can cause fatal fluid build-up in the lungs.

It is essential that these clots be dissolved or broken up in order to restore circulation, without causing massive bleeding in other areas not directly affected by the clot. If the clot is large enough and lodged in a blood vessel large enough for surgical intervention, the vascular surgeon can remove that clot by direct surgery or surgical devices. However, some smaller pieces of the clot may break off and not be captured. They may flow to the distal portions of the blood circulation and can cause an obstruction there.

The other method is to dissolve the clots by biochemical means. The prior art of treatment in these vaso-occlusive events involves mainly the administration of agents that will promote the dissolving of the already-formed clots, and/or prevention of the further formation (or enlargement) of clots. One agent is heparin, which is administered with the hope of preventing the formation of new clots while allowing the body's own mechanism of thrombolysis (destruction of the thrombus or clot) to take care of the already-formed clots. But heparin is not always effective against clots that are already formed and heparin can sometimes create problems with platelets, producing a situation called heparin-induced thrombocytopenia.

Other agents such a urokinase or TPA (tissue plasminogen activator) can actively dissolve clots. These agents are administered as a solution. After entry into the body, the enzyme molecules are distributed all over the body and not specifically targeted to the clot. Therefore, there is always the danger of causing bleeding in other areas which not directly affected by the occlusive clot(s). When a patient is treated for vaso-occlusion and the treatment leads to overall bleeding, the physician faces a very difficult decision, because further attempts to dissolve the clot will increase bleeding, while attempts to decrease bleeding (elsewhere) will typically worsen the original occlusive state. Most patients do not do well when this happens.

Therefore, there is a great need to have a product which will target the clot, so that (a) the pharmacological effects of clot-dissolving agents will not spread to unwanted locations but will focus mainly on the clot, and can (b) dissolve the clot at faster speed than soluble thrombolytic agents.

In cases involving infectious agents that can cause platelet clots in the lung leading to severe lung damage, the prior art is focused on using anti-infectious agents to kill the infectious agent and not the immediate harm done by the infectious agent in the target organ. A list of potential drugs useful to treat viral infection has been published. The list was included in “Antimicrob Agents Chemother. 2020 Mar. 9. pii: AAC.00399-20. doi: 10.1128/AAC.00399-20.” The title was “Compounds with therapeutic potential against novel respiratory 2019 coronavirus.” The author Martinez M A stated: “Currently, the expansion of the novel human respiratory coronavirus (known as: SARS-CoV-2, COVID-2019, or 2019-nCoV) has stressed the need for therapeutic alternatives to alleviate and stop this new epidemic. The previous epidemics of high-morbidity human coronaviruses, such as the acute respiratory syndrome coronavirus (SARS-CoV) in 2003, and the Middle East respiratory syndrome corona virus (MERS-CoV) in 2012, prompted the characterization of compounds that could be potentially active against the currently emerging novel coronavirus SARS-CoV-2. The most promising compound is remdesivir (GS-5734), a nucleotide analog prodrug currently in clinical trials for treating Ebola virus infections. Remdesivir inhibited the replication of SARS-CoV and MERS-CoV in tissue cultures, and it displayed efficacy in non-human animal models.” The author then discussed the use of a combination of the human immunodeficiency virus type 1 (HIV-1) protease inhibitors, lopinavir/ritonavir, and interferon beta (LPV/RTV-INFb) and concluded that “Therapeutics that target the coronavirus alone might not be able to reverse highly pathogenic infections. “A need exists for a new and novel thrombolytic microspheres that can be used to dissolve vaso-occlusive clots. In this regard, the present technology substantially fulfills this need. In this respect, the thrombolytic microspheres according to the present technology substantially departs from the conventional concepts and designs of the prior art, and in doing so provides an apparatus primarily developed for the purpose of dissolving ye vaso-occlusive clots.

### SUMMARY

In view of the above disadvantages, the present technology provides a novel thrombolytic microsphere to dissolve vaso-occlusive clots, and overcomes one or more of the mentioned disadvantages and drawbacks of the prior art. As such, the general purpose of the present technology, which will be described subsequently in greater detail, is to provide a new and novel thrombolytic microspheres to dissolve vaso-occlusive clots and method which has all the advantages of the prior art mentioned heretofore and many novel features that result in a thrombolytic microspheres to dissolve vaso-occlusive clots which is not anticipated, rendered obvious, suggested, or even implied by the prior art, either alone or in any combination thereof.

According to one aspect, the present technology can include a thrombolytic microsphere configured to dissolve vaso-occlusive clots. The microsphere can comprise a submicron albumin sphere, and an enzymatically active bioreactive agent encapsulated in the albumin sphere. The albumin sphere can be configured to dissolve vaso-occlusive clots in vivo.

According to another aspect, the present technology can include a composition for dissolving vaso-occlusive clots in a subject in need thereof. The composition can comprise a therapeutically effective amount of an albumin nanoparticle suspension containing submicron albumin spheres, and an enzymatically active bioreactive agent encapsulated in the albumin spheres. The albumin spheres can be configured to dissolve vaso-occlusive clots in vivo.

According to yet another aspect, the present technology can include a method of dissolving vaso-occlusive clots in a subject in need thereof. The method can include the steps of administering a therapeutically effective amount of an albumin nanoparticle suspension containing submicron albumin spheres, and an enzymatically active bioreactive agent encapsulated in the albumin spheres to the subject. The albumin spheres can be configured to dissolve vaso-occlusive clots in vivo.

In some or all embodiments of the present technology, the enzymatically active bioreactive agent can be urokinase.

In some or all embodiments of the present technology, the submicron albumin sphere can be a plurality of submicron albumin spheres contained in an albumin nanoparticle suspension.

In some or all embodiments of the present technology, the albumin nanoparticle suspension can be prepared by combining a pre-mixture including urokinase and a first human serum albumin with a bulk material including a second human serum albumin, glutaraldehyde and ethyl alcohol.

In some or all embodiments of the present technology, the albumin nanoparticle suspension can further include fibrinogen.

In some or all embodiments of the present technology, the fibrinogen can be a solution of fibrinogen and sodium tetradecyl sulfate.

In some or all embodiments of the present technology, the enzymatically active bioreactive agent can be urokinase.

Some or all embodiments of the present technology can include the step of preparing the albumin nanoparticle suspension by combining a pre-mixture including the urokinase and a first human serum albumin with a bulk material including a second human serum albumin, glutaraldehyde and ethyl alcohol.

In some or all embodiments of the present technology, the ethyl alcohol can be added to the bulk material as a first ethyl alcohol portion and a second ethyl alcohol portion different to that of the first ethyl alcohol portion.

In some or all embodiments of the present technology, the first human serum albumin can be at 2%, and the second human serum albumin can be at 6%.

In some or all embodiments of the present technology, the second human serum albumin can be at a volume of 100 uL, the glutaraldehyde can be at a volume of 50 uL, the first ethyl alcohol portion can be at a volume of 110 uL, and the second ethyl alcohol portion can be at a volume of 200 uL.

Some or all embodiments of the present technology can include the step of adding a solution of fibrinogen and sodium tetradecyl sulfate the albumin nanoparticle suspension after combining the pre-mixture and the bulk material.

In some or all embodiments of the present technology, the administering of the albumin nanoparticle suspension can be intravenously.

There has thus been outlined, rather broadly, features of the present technology in order that the detailed description thereof that follows may be better understood and in order that the present contribution to the art may be better appreciated.

Numerous objects, features and advantages of the present technology will be readily apparent to those of ordinary skill in the art upon a reading of the following detailed description of the present technology, but nonetheless illustrative, embodiments of the present technology when taken in conjunction with the accompanying drawings.

As such, those skilled in the art will appreciate that the conception, upon which this disclosure is based, may readily be utilized as a basis for the designing of other structures, methods, and systems for carrying out the several purposes of the present technology. It is, therefore, that the claims be regarded as including such equivalent constructions insofar as they do not depart from the spirit and scope of the present technology.

These together with other objects of the present technology, along with the various features of novelty that characterize the present technology, are pointed out with particularity in the claims annexed to and forming a part of this disclosure. For a better understanding of the present technology, its operating advantages and the specific objects attained by its uses, reference should be made to the accompanying drawings and descriptive matter in which there are illustrated embodiments of the present technology. Whilst multiple objects of the present technology have been identified herein, it will be understood that the claimed present technology is not limited to meeting most or all of the objects identified and that some embodiments of the present technology may meet only one such object or none at all.

### BRIEF DESCRIPTION OF THE DRAWINGS

The present technology will be better understood and objects other than those set forth above will become apparent when consideration is given to the following detailed description thereof. Such description makes reference to the annexed drawings wherein:

FIG. 1 is a diagram highlighting the interaction between enzymes that can promote the lysis of thrombi.

FIG. 2 is a graphical view of the activity of spheres made without human serum albumin (HAS) in the pre-mixture (unprotected UK) of the present technology.

FIG. 3 is a graphical view of the activity of spheres made with HSA2% in the pre-mixture (protected UK) of the present technology.

FIG. 4 is a tabular view of Table 4 showing an effect of different steps on the AP activity in the final suspension.

### DETAILED DESCRIPTION OF THE EMBODIMENTS

In the following description, for purposes of explanation and not limitation, specific details are set forth, such as particular embodiments, procedures, techniques, etc. in order to provide a thorough understanding of the present technology. However, it will be apparent to one skilled in the art that the present technology may be practiced in other embodiments that depart from these specific details.

The present technology can be implementable as a product and/or method to treat patients infected with respiratory viruses, such as but not limited to coronavirus, and in relieving hypoxic condition with the possibility to advert the need for intubation of a patient.

The present technology can include a suspension of thrombolytic albumin nanospheres that can target vaso-occlusive clots in vivo and then lyse the obstructive blood clots with high efficiency. Since the active enzyme is targeting and binding essentially only to obstructive clots, there is minimal risk of side-effects to the patient. The nanospheres are less than one micron in diameter, resulting in a size much smaller than even red blood cells.

One inspiration for the present technology results from the observation that respiratory viruses can damage the endothelium of the blood vessels in the lung, causing platelet aggregation there which will lead to build up of pressure and then the leakage of fluids from the intravascular system into the air sacs, thus drowning the patient internally. It can be appreciated that timely dissolution of these clots would reduce the need to intubate these patients and even allow time for the patient to reverse the pathology from the virus.

The thrombolytic albumin nanospheres of the present technology can work to dissolve clots and not to form co-aggregates with activated platelets to solidify clots. The present technology can nanospheres can still have fibrinogen on the surface to target (get trapped by) clots, but the interior of the nanospheres can carry enzymes such as, but not limited to, tPA or urokinase. The nanospheres can be porous and so plasma plasminogen can pass through, which will be converted by the urokinase inside the spheres to plasmin. Plasmin can then exit the nanospheres by diffusion and dissolve the clot that entraps the spheres.

Various known drugs such as tPA and urokinase (soluble forms) may be used to dissolve clots in vivo. However, a disadvantage of these known drugs is that these soluble enzymes will be distributed throughout the blood and washed out quickly, while the nanospheres of the present technology can be concentrated at the targeted site and the urokinase inside the nanospheres will continue to work there without further dilution until the clot is dissolved. Then the nanospheres will go elsewhere where there is a clot and work there.

The intactness of the vascular system is absolutely essential to life. In order to maintain its intactness, the vascular system of the body (the blood vessels and the blood) has two distinct activities which can take place in two phases (with respect to time): the immediate response to a wound or a cut in the blood vessel is to stop the bleeding. Then after some time has passed, and the blood vessels have healed, any clots that remain that still obstruct the flow of blood within the lumen of the blood vessel will be dissolved. In order to the perform these two different functions effectively, the body has at least two components (with respect to activity), one is a soluble fraction such as the coagulation factors (or the thrombolytic system); the other a cellular fraction which is mainly the platelets.

The first phase regards the intactness of structure: the immediate response to a wound or cut in the blood vessel is the formation of a therapeutic clot to seal the “hole” in the pipeline (the blood vessel) so that blood volume is not compromised. This clot in vivo is typically a mixture of the products from the coagulation factors and the cellular elements. The second phase to preserve the intactness of the system regards its function: any clots that result in the obstruction of flow (in addition to sealing off the hole in the wall of the pipeline) will need to be dissolved (eventually.)

There are at least three physiological reasons why the thrombolytic system (remove the clot) of the body is slower than the hemostatic system (stop bleeding). (a) It takes time for the leak or the hole in the wall of the blood vessel to heal. Therefore, the thrombolytic process will take place essentially only after the hole is healed. Otherwise bleeding will occur again through the same damaged area. (b) Sometimes the clot is not therapeutic but is harmful, but the occlusion builds up gradually such as a coronary build-up of cholesterol plaques. The partial obstruction may cause symptoms such as angina, but the obstruction is not immediately fatal, unless the clot expands. The body can respond by creating new collateral blood circulation to bypass the obstructed vessel. Meanwhile the obstruction stays. (c) The circulatory system often has more than one blood vessel feeding the tissues downstream—this is known as the body's natural collateral circulation. Therefore, even if an obstruction occurs suddenly and severely, there is still some flow to the downstream tissues and therefore the clot is not immediately removed—the thrombolytic system is often delayed—so as to avoid unintended bleeding elsewhere. The balance between hemostasis and thrombolysis is very delicate. In pathological conditions, the balance is “out of balance” but it is hard to put it back to balance pharmacologically.

Regarding the formation of a clot in vivo to seal off any “hole” in the wall of the blood vessel: the first component is the liquid phase of the blood (the plasma) which contains dissolved proteins including the coagulation factors. When a blood vessel is cut or a wound appears on the internal wall (the endothelium) of the blood vessel, the precursors of these coagulation factors will be converted into the active form and these activated factors will develop a clot, which would ideally seal off the wound in time so that the minimal amount of blood is lost from the intravascular compartment (all that is inside the blood vessels) to the extravascular compartment (all that is outside the blood vessels, including muscles or the space outside the body.) However, it takes time for a firm clot to be formed, sometimes requiring many seconds, if not minutes.

The second component important in the formation of a clot in vivo is the cellular fractions of the blood. There are three major types of cells in the blood, two of which are not directly involved in hemostasis (I.e. control of bleeding): the red cells are responsible for oxygen delivery; the white cells for defense against infection. The cells responsible for hemostasis are the platelets. When an injury occurs, the exposed collagen at the wound would trigger the conversion of the circulating (non-activated) platelets into activated platelets. This reaction can occur within milli-seconds, which means the platelets will change from non-sticky to very sticky within several thousandth of a second. This rapid response is important because the blood cells travel at a high speed within the blood vessels, being propelled by the squeeze from the heart. If the conversion is any slower, the sticky platelets would be sticking to a surface too far downstream from the wound to be able to attach to the wound to seal off the wound.

It is now understood that the key step of activation of platelets is the binding of fibrinogen onto the platelet surface. Fibrinogen is a coagulation factor (also known as Factor I). Therefore, there is co-operation of the liquid phase (the dissolved coagulation factors) and the cellular component during activation of the platelets. Even so, it is obvious that a preformed mass such as the platelet would be more effective in sealing off a wound, like the greater effectiveness of rocks (preformed solids) fixing the leak in a dam than wet cement (the coagulation factors needing time to solidify.)

It should be noted that a clot formed at the wound site of the endothelium to seal off the wound without obstructing the flow of blood is to be understood as a “therapeutic clot.” It is to be distinguished from “obstructive clots” which block the flow of the blood within the blood vessels. Any medical intervention to assist the hemostatic function of the patient must not contribute to the formation of obstructive clots. Any medical intervention to reduce the size of an obstructive clot should ideally reduce the size of the existing clot without causing bleeding elsewhere.

Yen has disclosed numerous prior arts related to albumin nanospheres which serve to promote hemostasis, e.g. “biologic devices for hemostasis” U.S. Pat. No. 9,114,127 issued Aug. 25, 2015. In contrast, this patent is about “thrombolytic microspheres” which will perform the opposite function, i.e. the dissolution of clots.

In this disclosure, the term “microsphere” is used generically to mean protein spheres which can have diameters in the micron range, but it can include in the preparation some smaller spheres in the nanometer range. The term “nanosphere” is also used generically to mean protein sphere which have diameters less than one micron. It is possible that in some medical applications, larger spheres in the micron range (microspheres) may be more effective than nanospheres because rheologically larger particles flow near the center of the blood vessels where the obstructive clots may be more likely to be located (while smaller particles circulate near the endothelial wall.)

Due to the aim of the present technology, which is aimed at dissolution of undesired and harmful obstructive clots, the term “clots” used in this disclosure will refer mainly to obstructive clots which are harmful to the patient and not “therapeutic clots” unless specified. More importantly the thrombolytic microspheres of the present invention will be designed with the capacity of targeting obstructive clots—they will stick to these clots for the purpose of providing agents locally which will dissolve the harmful clots specifically and quickly, without causing bleeding side effects elsewhere (such as can easily occur with thrombolytic agents which are soluble agents.)

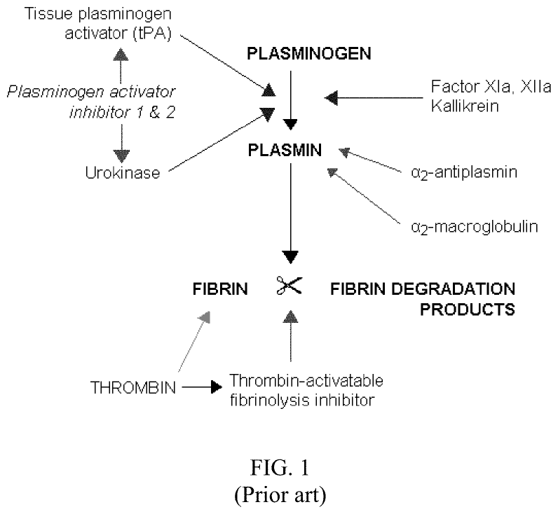

Regarding the enzymes that can promote the lysis of thrombi, the following diagram shown in FIG. 1 highlights the interaction between these enzymes. The diagram has been published: by Jfdwolff at en.wikipedia, CC BY-SA 3.0.

The blue arrows in the diagram indicates stimulation of the process, while the red arrows denotes inhibition.

It can be seen from the diagram that plasmin is the key enzyme in breaking down fibrin which is the main component of a clot. Fibrin itself is formed by the breakdown of fibrinogen by the activity of thrombin. It can also be noticed that the enzyme plasmin is also tightly regulated by both activating enzymes (blue arrows) as well as inhibiting enzymes (red arrows.) The regulation has to be very tight because the body cannot afford to have “run-away” enzyme reactions, i.e. the body cannot afford to have pathways that promotes itself in a unidirectional way. In other words, if there is too much activity leading to clot-formation, the body will turn on the clot-lysis pathways. If, however, the clot-lysis forces are too strong, the body will turn on the clot formation pathways. All these are needed to preserve the health of the vascular system.

There exist in the blood several enzymes that can convert plasminogen to plasmin. One of them is tPA (tissue plasminogen activator) so called because it is released by the tissue near a healed wound. The tPA itself can have at least three subsequent pathways, depending on the need of the body at that time. Therefore, if tPA were to focus on or continue towards its thrombolytic duty (versus its other fates) it should be protected from the inhibition processes in the blood. Most advantageous to a continuous process of thrombolysis will be the immobilization of tPA at the site of the unwanted clot. Other enzymes such as urokinase would also need to be concentrated at the unwanted clot site so that the soluble plasminogen molecules in the blood reaching the clot can be locally converted into plasmin which is most needed at that time at that site.

A short description on the fate of tPA is as follows:

“following administration and release, tPA can be absorbed by the liver and cleared from the body through receptors present therein. One of the specific receptors responsible for this processes is a scavenger protein, specifically the LDL Receptor-Related Protein (LRP1). tPA additionally can be bound by a plasminogen activator inhibitor (PAI), resulting in inactivation of its activity, and following clearing from the body by the liver. Lastly, tPA can bind plasminogen, cleaving off the bound plasmin from it. Plasmin, another type of protease, can either be bound by a plasmin inhibitor, or work to degrade fibrin clots, which is the highest utilized and desired pathway.”

While the formation of therapeutic clots at a wound site in the endothelial wall is a normal event and the formation of occlusive clots in a large wound in certain parts of the body during a trauma is unavoidable, healthy patients tend not to suffer excessive harm from these processes. However, in some patients, the abnormal situation will arise: the formation of unwanted clots is a recurrent and pathological problem. Recent discoveries have shown that the process is complex and that some white cells called neutrophils may be involved.

In a paper titled “Neutrophil extracellular traps in immunity and disease” the author Dr. Papayannopoulos stated in the abstract: “Neutrophils are innate immune phagocytes that have a central role in immune defense. Our understanding of the role of neutrophils in pathogen clearance, immune regulation and disease pathology has advanced dramatically in recent years. Web-like chromatin structures known as neutrophil extracellular traps (NETs) have been at the forefront of this renewed interest in neutrophil biology.” (Nature Reviews Immunology vol 18, pages 134-147, 2018).

Neutrophil Extracellular Traps (NETS) were initially thought to be DNA material extruded from the white cells for the purpose of entrapping bacteria. However, additional (wanted and unwanted) functions of NETS are soon discovered, including “NETs can also occlude the vasculature by promoting thrombosis and obstruct important organ areas, capture metastatic tumors and delay wound healing in diabetes.” Therefore, the timely destruction or removal of NETS may contribute to the faster recovery of a large number of patients suffering from these conditions.

In fact, platelets are involved in the promotion and formation of NETS. In an article by Dr. Stephen Clark et. al, titled “Platelet TLR4 activates neutrophil extracellular traps to ensnare bacteria in septic blood” the introduction stated: “It has been known for many years that neutrophils and platelets participate in the pathogenesis of severe sepsis, but the interrelationship between these players is completely unknown. We report several cellular events that led to enhanced trapping of bacteria in blood vessels: platelet TLR4 detected TLR4 ligands in blood and induced platelet binding to adherent neutrophils. This led to robust neutrophil activation and formation of neutrophil extracellular traps (NETs). Plasma from severely septic humans also induced TLR4-dependent platelet-neutrophil interactions, leading to the production of NETs. The NETs retained their integrity under flow conditions and ensnared bacteria within the vasculature. The entire event occurred primarily in the liver sinusoids and pulmonary capillaries, where NETs have the greatest capacity for bacterial trapping. We propose that platelet TLR4 is a threshold switch for this new bacterial trapping mechanism in severe sepsis.” (NATURE MEDICINE VOLUME 13, NUMBER 4, APRIL 2007). Note: TLR4 means “Toll-like receptor 4”.

Therefore, in this disclosure, the term “vasculature” includes also the various locations in the body not often associated with the “intravascular compartment” but which are obviously connected to or associated with it, such as the “liver sinusoids”, the pulmonary capillaries and other sinusoids such as those within the spleen or similar structures.

The identification of molecules that modulate the release of NETs has helped to refine our view of the role of NETs in immune protection, inflammatory and autoimmune diseases and cancer. Here, I discuss the key findings and concepts that have thus far shaped the field of NET biology.

The authors pointed out that neutrophils can extrude their nuclear material which is normally used to trap bacteria. But under abnormal conditions, massive clots are formed which will obstruct the flow of blood.

Another major application of the present invention is the treatment of severe ill patient after their infection with infectious agents that harm their lungs. The agent can be viral, such as the common flu virus or the corona virus. Alternatively, the agent can be a bacterial agent, or a combination of viral infection superimposed with a bacterial infection.

Dr. Susan Armstrong and her colleagues in Toronto studied the effect of influenza infection on the lung microvascular endothelium. The data was published in PLoS One. 2012; 7(10): e47323. The title was “Influenza Infects Lung Microvascular Endothelium Leading to Microvascular Leak: Role of Apoptosis and Claudin-5.” The scientists noted that “Severe influenza infections are complicated by acute lung injury, a syndrome of pulmonary microvascular leak.” Although the pathogenesis of this complication is unclear, the investigators hypothesized that human influenza could directly infect the lung microvascular endothelium, leading to loss of endothelial barrier function. The data showed that the hypothesis was correct.

The severity of a double infection by the influenza virus plus bacteria was studied. The work was published in Am J Respir Cell Mol Biol. 2015 October; 53(4):459-70. Title “Influenza-Induced Priming and Leak of Human Lung Microvascular Endothelium upon Exposure to Staphylococcus aureus” the authors (Wang C et al) made the following statements: “Respiratory failure after superinfection presents as acute respiratory distress syndrome, a disorder characterized by lung microvascular leak and edema. The objective of this study was to determine whether the influenza virus sensitizes the lung endothelium to leak upon exposure to circulating bacterial-derived molecular patterns from Staphylococcus aureus.” The data showed that indeed, “Influenza virus primes the lung endothelium to leak, predisposing patients to acute respiratory distress syndrome upon exposure to S. aureus.”

Another study concerns the role platelets, published in Am J Respir Crit Care Med. 2015 Apr. 1; 191(7):804-19. doi: 10.1164/rccm.201406-1031OC. The title is “Platelet activation and aggregation promote lung inflammation and influenza virus pathogenesis.” the authors (Le V B et al) noted that “Lungs of infected mice were massively infiltrated by aggregates of activated platelets. Platelet activation promoted influenza A virus pathogenesis. Activating protease-activated receptor 4, a platelet receptor for thrombin that is crucial for platelet activation, exacerbated influenza-induced acute lung injury and death. In contrast, deficiency in the major platelet receptor glycoprotein Ma protected mice from death caused by influenza viruses, and treating the mice with a specific glycoprotein IIb/IIIa antagonist, eptifibatide, had the same effect. Interestingly, mice treated with other antiplatelet compounds (antagonists of protease-activated receptor 4, MRS 2179, and clopidogrel) were also protected from severe lung injury and lethal infections induced by several influenza strains.” The investigators concluded that “The intricate relationship between hemostasis and inflammation has major consequences in influenza virus pathogenesis, and antiplatelet drugs might be explored to develop new anti-inflammatory treatment against influenza virus infections.”

The role of platelet aggregation and adhesion to the lung tissues was studied. The data was published in J Virol. 2016 Feb. 15; 90(4): 1812-1823. The title was “Influenza Virus Infection Induces Platelet-Endothelial Adhesion Which Contributes to Lung Injury.” The authors (Michael G. Sugiyama et al) stated the following: “Despite annual vaccination programs and widely available antiviral drugs, seasonal influenza alone causes an estimated tens of thousands of deaths in North America annually. Most deaths occur due to pulmonary complications, such as primary viral pneumonia or a superimposed bacterial pneumonia. In both, respiratory deterioration is marked by acute lung injury, a potentially fatal syndrome of pulmonary edema that occurs due to the increased permeability of the lung microvasculature. Therapeutic options are limited. While antiviral drugs exist, they only partially reduce mortality, they must be administered early to be maximally effective, and their use is complicated by the rapid development of resistance. For instance, almost 100% of H3N2 strains of influenza A are already resistant to amantadine. Thus, new therapies for these most severe cases of influenza are urgently needed.”

Furthermore, the authors noted: “Importantly, whether viral infection affects the relationship of the lung endothelium with other cells in the lung, such as platelets, is unknown. This is an important question since platelets have been identified to contribute to certain forms of lung injury through their recruitment of leukocytes and induction of an inflammatory response. Importantly, the endothelium normally is antithrombogenic and prevents platelet adhesion. An effect of the virus to induce platelet-endothelial adhesion is plausible: numerous reports describe pulmonary thrombi as an important complication of severe infections with influenza, while endothelial activation, which would be expected to induce platelet adhesion, was highly correlated with death from influenza in a murine model. Furthermore, a study of patients with venous thromboembolism found that vaccination against influenza was associated with a protective effect against pulmonary clots. Determining how influenza infection affects platelet-endothelial interactions may have important clinical implications, since numerous platelet antagonists are available already and might constitute useful therapies. “However, the authors did not teach how to use these “numerous platelet antagonists” and none of the agents on the markets have the capacity to target the platelet clots in the lung and dissolve them.

Experiment One: Can Fibrinogen-Coated Albumin Spheres (FAS) Bind Additional Bioreactive Agents onto the Spheres?

### Introduction

(a) The enzyme Alkaline Phosphatase (AP) will be used as an example of a bioreactive agent because its activity in the soluble form or after incorporation into a sphere can be measured easily. AP that are active (not inactivated by the process of incorporation onto a sphere) can catalyze a reaction with a “phosphatase substrate” which will result in a yellow color that can be measured in a spectrophotometer.

(b) Other useful agents will include enzymes which will stimulate the formation of thromolytic (or fibrinolytic) agents such as plasmin (from plasminogen), including tPA, urokinase, Factor Xia, XIIa, and Kalikrein. Another category of bioactive agent is DNAase, which are enzymes that will “cut” DNA strands, some for double-stranded DNA, some for single-stranded DNA, some at specific DNA sequences, some at the terminal sites, etc. These will be instrumental in the destruction of the scaffolds within the NET obstructive diseases.

(c) The bioreactive agent will be directly added to the FAS to see if the bioreactive agent can bind directly to the spheres due to the following observations: 1. In the blood the concentration of fibrinogen and albumin are 2 mg/mL and about 40 mg/mL, respectively. There is no evidence that they are bound to each other, i.e. the two molecules are in freely soluble states. Yet when a solution of fibrinogen (about 1.4 mg/mL) is added to blank spheres (containing about 8 to 10 mg spheres/mL) in a 1:3 ratio (v/v) the fibrinogen molecules binds essentially completely onto the spheres. There is no evidence that the fibrinogen molecules can detach easily from the spheres. The attachment is non-covalent and is probably based on molecular attraction between the fibrinogen molecules and the albumin spheres. This experiment evaluates if there is a similar mechanism of attachment between AP and the spheres, even though the spheres already have some fibrinogen occupying some “attachment sites.”

### Materials and Methods:

Alkaline Phosphatase (P-7640) and the substrate are purchased from Sigma Aldrich. The FAS used is Lot2018FEB01FPS which is the sterile product that has been tested and shown to be effective in animal studies. A stock solution of AP was prepared at 1 mg/mL, which is then serially diluted to 0.3, 0.1, 0.03 mg/mL. Similarly, the FAS stock suspension has 8 mg spheres/mL, which was serially diluted to 2.7, 0.8 mg/mL in water. All the (diluted) AP solution were then mixed with the various concentrations of FAS for 4 hours at room temperature to allow (any) attachment of AP to the spheres. The mixture of AP plus sphere was then divided into two portions: (a) the first portion is the WS (Whole Suspension) which is the supernatant plus sphere fraction; (b) the second portion is centrifuged to remove the spheres, leaving the S (Supernatant) fraction. Two controls were included: one is without the spheres (but with AP solution); the other is without the AP (but with the spheres) The various fractions were then assayed for phosphatase activity using the standard photometric methods (read Optical Density at 405 wavelength).

The hypothesis is: Some AP should stick to the FAS. Therefore, the phosphatase activity in the WS should be higher than the S fraction (where the activity attached to the spheres had been removed.) The amount of AP attached to the spheres should be equal to the activity in WS—activity in S.

### Results:

After correcting for (by removing) the OD of the controls, the OD of all the tubes for WS is the same as the OD in the S (regardless of what concentration of spheres is mixed with what concentration of AP). This shows clearly that the activity in the sphere fraction (which is WS-S) in all cases is zero.

### Conclusion:

There is no evidence that any AP enzyme has attached to the spheres. The hypothesis that AP should stick to the spheres (just like fibrinogen would) is false. Therefore, the next step is to test if AP enzymes can attach to blank spheres or whether AP can be incorporated into the spheres during the manufacturing steps of making the spheres.

Experiment Two: Can Freshly-Made Blank Albumin Spheres (AS) Bind Additional Bioreactive Agents onto the Spheres?

### Materials and Methods:

Freshly manufactured blank spheres were prepared as follows: 30 mL of human serum albumin (bought from Octapharma) at 5.5% is mixed with 15 mL of GL-1 (glutaraldehyde solution at 0.15 mg/mL in water). After incubation for 60 min and 150 min respectively, an alcohol solution (75% Ethyl alcohol containing glutaraldehyde 0.5 mg/mL) was added, volumes being 39 mL and 63 mL. The turbidity of the suspension indicated that spheres have been formed. After 2 hours of stabilization (to prevent re-solubilization of the spheres when the alcohol concentration is decreased by the addition of later solutions) the turbid suspension was subdivided into smaller portions for the mixing with various concentration of AP solutions (in essentially the same sequence as in the preparation of fibrinogen-coated albumin spheres.)

Subsequently, the phosphatase activity of the WS and the S fractions in various tubes were assayed as described in Experiment One.

### Results:

Microscopic examination of the sphere suspensions mixed with AP solution showed no aggregates or spheres larger than 5 microns. This means the addition of AP solutions (which contain some unknown salts) had no adverse effect on the stability of the spheres formed under these conditions.

After correcting for (by removing) the OD of the controls, the OD of all the tubes for WS is the same as the OD in the S (regardless of the concentration of AP mixed with the blank sphere suspension). This shows clearly that the activity in the sphere fraction (which is WS-S) in all cases is zero.

### Conclusion:

There is no evidence that any AP enzyme has attached to the (blank) spheres. The hypothesis that AP should stick to the spheres (just like fibrinogen would) is false. Therefore, the next step is to test if AP enzymes can be incorporated into the spheres during the manufacturing steps of making the spheres.

Experiment Three: Can Bioreactive Agents be Incorporated into Albumin Spheres (AS) by Adding the Bioreactive Agent to the Human Albumin Solution Before the Subsequent Steps of Making Spheres?

### Materials and Methods:

The method of making spheres here follows the similar steps described in Experiment Two, except AP was first added to the Human Serum Albumin (5.5%) instead of adding the AP to the suspension containing the blank spheres.

### Results:

Microscopic examination of the suspensions A, B, C, D showed that the presence of AP with some unknown amount of salt present in the AP solution does not appear to adversely affect the formation of the spheres, as illustrated in Table 1. All the spheres are substantially smaller than 0.5 micron.

TABLE 1

| | | | | GL1 + other | Presence of |
|---|---|---|---|---|---|
| | Albu- | Phos- | | solutions | aggregates or |
| | min | phatase | | to make | spheres larger |
| tube | 5.5% | 5 mg/mL | water | spheres | than 0.5 micron? |
| A | 2 mL | 0.2 mL | 0 mL | 8.8 mL | No |
| B | 2 mL | 0.07 mL | 0.13 mL | 8.8 mL | No |
| C | 2 mL | 0.02 mL | 0.18 mL | 8.8 mL | No |
| D | 2 mL | 0 | 0.2 mL | 8.8 mL | No |

Aliquots of the suspensions A, B, C, D were divided into WS and S fractions and their phosphatase activity measured. Tube D served as the control because it has spheres but no AP. The data showed again that the WS fractions of all the tubes from A, B, C were the same as the AP activity in the S fraction (not shown here). This means there is no incorporation of AP into the spheres regardless of the amount of AP added to the albumin 5.5% solution. All the AP activity resides with the supernatant fraction.

EXPERIMENT FOUR: Will “prelinkage” of the bioreactive agent with HSA (human serum albumin) promote the incorporation of the bioreactive agent into albumin spheres (AS)?

### Introduction

In a previous disclosure U.S. Pat. No. 5,059,936 “Manufacturing protein microspheres” (issued on Dec. 3, 1991) the author Yen might have listed a number of enzymes such as Alkaline Phosphatase as potential candidates for incorporation into albumin spheres.

However, there is no teaching there how each different enzyme will need to be treated before they can be incorporated into the spheres. The term “incorporation” means essentially that the bioreactive agent is attached or linked to the spheres, without specifying whether the agent is mainly on the surface or in the interior of the spheres.

Given the data in Experiment Three, that adding AP to the HSA5.5% directly before the addition of GL-1 (glutaraldehyde at 0.15 mg/mL) did not result in the incorporation of the (free) AP into the spheres, the idea for Experiment Four is to “pre-link” the AP molecules to HSA in a step separate from the addition of the HSA5.5%, so that the AP is not a molecule independent of albumin but is part of a conjugate with albumin. The purpose is to “capture” the AP as the albumin-moiety of the conjugate is caught up during the formation of the sphere with other free albumin molecules (provided by the HSA5.5%)

This experiment used a range of concentration of HSA (from 0, i.e. water, to 10 mg HSA per mL) to assess if the amount of HSA in the “pre-link” mixture has an effect on the capacity of the system to incorporate AP into the spheres.

### Materials and Methods:

The method of making spheres here follows the similar steps described in Experiment Two, except AP was first added to a small volume of Human Serum Albumin (not 5.5% but 0 to 10 mg/mL) with the potential of forming an AP-albumin conjugate, by mixing in GL-2 (which is not 0.15 mg/mL but 0.5 mg/mL). Then a larger volume of HSA5.5% is added to provide the bulk material to form spheres.

### DETAILED DESCRIPTION

A volume (50 uL) of AP (5 mg/mL) was mixed with an equal volume (50 uL) of HSA at various concentrations, as indicated (tubes P, Q, R, T, W) in the table below. Tube W was the control where 50 uL of water is used and no AP. Then 50 uL of GL-2 (0.5 mg/mL) was added to all tubes potentially link the AP molecules to HSA molecules, for 1 hour at room temperature. After that, 1 mL of HSA (8%, not 5.5%) was added to the tube. The steps to form spheres were started with the addition of GL-1(0.15 mg/mL, 0.5 mL) followed by the addition of EG (75% Ethyl alcohol with 0.5 mg/mL of Glutaraldehyde) at 60 seconds and 150 seconds respective, at volumes of 1.3 ml and 2.1 mL, respectively. The suspension turned turbid immediately and spheres were observed under the microscope. To allow development of a yellow color suitable for the reading with the spectrophotometer, the suspensions were further diluted with water 5× before the alkaline phosphatase assays were performed.

A standard curve of AP was generated by reaction of known (serially diluted) concentrations (ug/mL) of AP with the phosphatase substrate to yield the various ODs.

### Results:

The standard curve of OD vs AP concentrations showed that the coefficient of extinction is 0.1096 OD per unit of AP activity (at 15 ug per mL). This value is used to covert the various OD of the various fractions to the “AP concentration” of the WS (whole suspension) or the S (Supernatant), as shown in Table 2.

TABLE 2
Effect of the concentration of HSA in the pre-link mixture on the AP activity in the sphere suspensions

| | Vol of | | | Expected AP | Measured | | Calculated |
|---|---|---|---|---|---|---|---|
| | AP stock | Conc of | Vol of | concentration | AP conc in | Measured | AP conc |
| | used, uL (at | HSA used, | other | in the total | the Whole | AP conc in the | in the |
| | 5 mg/mL) | (mg/ml) | reagents | volume (of | Suspension | Supernatant | Sphere |
| | in the pre- | In the pre- | used to make | 5.05 mL sphere | Fraction | Fraction | Fraction |
| Tube | link mix | link mix | spheres, mL | suspension), ug/mL | (WS) | (S) | (WS − S) |
| P | 50 | 10 | 4.9 | 49.5 | 53.1 | 39.9 | 13.3 |
| Q | 50 | 5 | 4.9 | 49.5 | 47.4 | 35.6 | 11.8 |
| R | 50 | 2.5 | 4.9 | 49.5 | 22.8 | 19.8 | 2.9 |
| T | 50 | 0 | 4.9 | 49.5 | 7.6 | 9.4 | −1.8 |
| W | 0 | 10 | 4.9 | 0 | 0 | 0 | 0 |

TABLE 3
Protein concentration of the various fractions

| | | | Sphere fraction, |
|---|---|---|---|
| | Whole Suspension | Supernatant (S), | mg/mL |
| Tube | (WS), mg/mL | mg/mL | (=WS − S) |
| P | 13.1 | 1.3 | 11.8 |
| Q | 12.5 | 1.4 | 11.2 |
| R | 12.8 | 1.5 | 11.3 |
| T | 13.0 | 1.3 | 11.7 |
| W | 14.3 | 1.2 | 13.1 |

The data in Table 3 show that sphere formation is not adversely affected by the step of “pre-linkage.” In contrast to the concentration of HSA used to make spheres (5.5%) previously, this experiment employed a higher concentration (8.0%) so that slightly larger spheres can result, with a larger mass each to more likely “capture” the presumed AP-HSA conjugate. The yield is about 11.8 (spheres)/13.1 (total protein) (using the value in Tube P) which is about 90%.

In terms of the AP activity (converted from the OD to concentration by using the coefficient of extinction obtained from the standard curve) in the various fractions, the data showed a surprising result. When the concentration of the HSA is 5 mg/ml (Tube Q) or above (e.g. 10 mg/mL in tube P), there is no loss of AP activity during the formation of the sphere. The concentration of AP in the final 5.05 mL of sphere suspension should be 250 ug divided by 5.05 mL, which is 49.5 ug/mL. This is about the same value obtained in the WS fraction of Tube P and Q (within experimental error of measurement.)

However, when the concentration of HSA in the pre-link mixture is below 5 mg/ml, such as in Tube R (2.5 mg/mL) or Tube T (water only), there is an obvious “inactivation” of the AP enzyme in the sphere suspension. The WS fractions in Tube R and T showed an equivalence of only 22.8 and 7.6 ug/mL of AP, respectively.

The activity of AP in the Supernatant fraction is similarly affected. The cause is unknown. Additional experiments (as shown below) does not indicate that it is an effect from alcohol, since all tubes have the same amount and concentration of alcohol.

The fraction of AP that is incorporated into the sphere fraction can be obtained by WS-S. This shows that about 13.3, 11.8 ug/mL of AP is incorporated when the HSA has sufficiently high concentration (such as in Tubes P and Q) but it drops off drastically when the HSA in the pre-link mix is low (such as in Tube R and T).

We have not evaluated the effect of using higher concentrations or higher ratios in volume of HSA in the pre-link mixture (against a known mass of AP). It is possible that higher than 13.3 ug/mL of AP can be incorporated under those new conditions.

### Discussion/Comments:

It should also be noted that the “mass of AP” (converted from the OD, which is the activity of the enzyme) that is bound to the “sphere fraction” is obtained by the “fraction of AP in WS” minus the “faction of AP in S”. This method has to be used because it is difficult to measure directly the AP activity from (or associated with) the spheres. Resuspension of the sphere pellet after centrifugation often does not produce single, unaggregated spheres (as in the original suspension before centrifugation.) These unevenly resuspended particles will cause disturbance in the light when the OD is measured, causing unreliable OD results. Also, some spheres will be stuck to the pipet and thus resulting in some loss of spheres during the re-suspension process which will produce variations between experiments.

It should also be noted that when the spheres were first formed, they were homogeneously suspended in the Supernatant phase. Centrifugation of 1 mL of the undiluted suspension will yield a sphere pellet of about 100 uL. This means the AP enzymes are highly concentrated in the spheres because the spheres by themselves occupy a much smaller volume than the Supernatant fraction. Referring to Tube P, where the AP activity in the WS and Sphere fraction is 39.9 and 13.3 ug/mL, respectively, correction of the “sphere volume” in the suspension will lead to a remarkably different result. It will offer a very different perspective. The AP activity in the WS fraction will remain the same: being 39.9 ug/mL volume, but the concentration of AP in the Spheres will become 133 ug per mL volume of spheres. Therefore, incorporation of any bioreactive agent into the spheres (regardless of whether the agent is on the surface or inside the interior of the spheres) will allow a proportionally faster reaction with the substrates of the reactive agent, due to the super-normal concentration of the agent packed on or within the sphere. This super-high concentration of the reactive agent is often not be reachable in the soluble state (above the saturation concentration.) Even if the super-high concentration can be reached, once a volume of the super-high concentration is injected into the blood, the concentration of the reactive agent will be diluted quickly. Only the spheres can bring that super-high concentration of the agent to the site where it can do the maximum good.

The mass of human serum albumin needed to promote pre-linkage is at least 50 uL×5 mg/ml for a mass of AP equal to 50 uL×5 mg/mL (tube Q). This means a ratio of 1:1 in terms of micrograms or milligrams. However, the molecular weight of HSA is 66.5 kDa verses that of AP being 160 kDa. That means at least 3 molecules of HSA must be provided per molecule of AP for the pre-link step to work in promoting the incorporation of AP via a potential conjugate with HSA. However, the exact nature of this interaction is not obvious because in Experiment 3 where the AP was mixed with HSA5.5%, the ratio of HSA to AP in terms of weight is 55 mg/mL×2 mL versus 5 mg/mL×0.2 mL, which is 110 mg of HSA per 1 mg of AP but the AP was not incorporated into the spheres. Therefore, the nature of this “conjugate” or its process of formation will be explored in the next experiment.

### Experiment Five: Which Step(s) in the Formation of Spheres Lead to the Inactivation of AP?

### Introduction

The term “inactivation” can mean any of the following, which can lead to the “inactivity” of the enzyme, whether temporarily or permanently. The inactivation may mean: (a) the destruction of the entire enzyme so that its tertiary structure is destroyed and the protein cannot perform its catalytic function; (b) only the active site is affected: being blocked or denatured in some way; (c) the enzyme buried in the interior of the sphere or on the surface of the spheres cannot be reached by the substrate (which is typically smaller in molecular weight and size than the enzyme) and therefore, there is the substrate in the mixture is not acted on; (d) other mechanisms of inactivation.

### Materials and Methods:

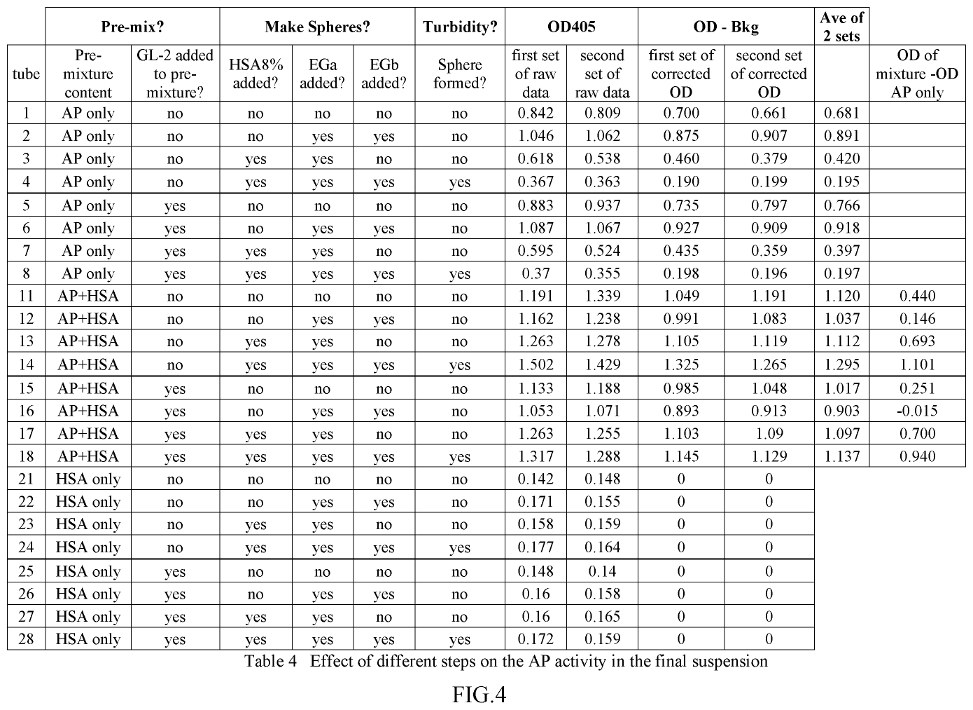

The overall description for the formation of a “conjugate” between albumin molecules and AP molecules has been described in Experiment Four. To investigate whether the slightly higher concentration of glutaraldehyde in GL2 (being 0.5 mg/ml, same as the concentration of glutaraldehyde in the EG solutions) has an effect, or the addition of EG (75% ethanol including 0.5 mg/ml of glutaraldehyde) may be detrimental to the AP activity, a variety of conditions were constructed. Table 4 listed the various conditions, as best illustrated in FIG. 4. Essentially, Tubes 1 to 8 contained only AP (25 uL at 4 mg/mL plus 25 uL of water) in the fake “pre-mixture” (no HSA). Tubes 11 to 18 contains the true “pre-mixture” (using a mixture of 25 uL of AP at 4 mg/mL and 25 uL of HAS at 20 mg/mL, not 10 mg/mL.) Tubes 21 to 28 contains the control mixture (the OD will reflect the background color of the AP substrate and variation in the OD due to any turbidity caused by spheres.) Then GL-2 was added to the mixtures (25 uL).

After incubation for one hour, various components to make spheres were added. In tubes marked “no” an equivalent volume of water was used as a substitute for that component (whether the component was HSA8% or EGa or EGb.) All tubes that have HSA8% will have GL-1 added at time zero to start the sphere-making process. EGa and EGb are similar solutions (75% ethanol containing 0.5 mg/mL of glutaraldehyde, volumes are 0.65 mL and 1.30 mL, respectively.) They are added in two portions because the sum of the volume of EGa and EGb when added into one volume would have created large spheres and often the formation of aggregates. After the addition of EGa (added at time equal to 60 seconds), the solutions will remain clear, meaning spheres have not been formed. However, there is enough alcohol in the mixture to have changed the components inside the tubes, as will be discussed in the Comments section below. Immediately upon the addition of EGb (added at time equal to 150 second) the previous-clear solutions will turn turbid and spheres can be observed under the microscope.

As can be noted in Table 4—as illustrated in FIG. 4, within the group of tubes 1 to 8, tubes 1-4 are parallel to tubes 5-8 except Tubes 1 to 4 had no GL-2. Within the tubes 1-4, Tube 1 and 2 had no HSA8% added, while Tube 3, 4 had the sphere-forming ingredient of HSA8% added. Tube 2 had both EGa and EGb added, to reveal with a high alcohol content (compared to tube 1) would have a severely adverse effect on the activity of AP. Tube 3 would have only EGa, while Tube 4 would have a full “complement” of the alcohol solution added which is needed to make spheres. It is a surprise that the content of Tube 3 is markedly different from Tube 4 (to be discussed below.)

### Results:

After properly dilution of the contents of all the tubes (to allow development of the yellow color within range of the spectrophotometer), an aliquot from each tube was used to react with AP-substrate to generate a yellow color. After the standard time of incubation, the reaction was stopped by the addition of sodium hydroxide solution. Then the content of all the tubes were centrifuged: this step will remove the spheres (if any were present) in order to eliminate any potential of disturbance to the optical paths when the OD of the supernatant was read in the spectrophotometer. Even though this experiment included a step of centrifugation to remove the spheres (in Tube 4, 8, 14, 18, 24, 28) the OD value is the AP activity of the WS because both sphere fraction and supernatant fractions were present when the phosphatase substrate was added. The yellow coloration from the product of the AP reaction was fully soluble and not pelleted, nor carried by the spheres to the bottom of the centrifuge tube.

After the OD of the various solutions/suspensions were measured and recorded, the OD were repeated to ensure the values are reliable. The raw results are displayed in the 8th and 9th column, respectively (First and Second set of raw data). The data show that the control set (Tubes 21 to 28) had a background color (from the phosphatase substrate) of about 0.142 to 0.177. The OD from this set of controls will be subtracted from the corresponding tubes from Tubes 1 to 8 and similarly from Tubes 11 to 18, to obtain the “corrected OD.”

The data showed that addition or absence of GL-2 made no difference whether AP is inactivated or not during the entire process. One can compare Tubes 1-4 with Tubes 5 to 8; or Tubes 11-14 with Tubes 15-18; or Tubes 21-24 with Tubes 25-28. For example, when the OD of Tubes 1 to 4 is compared with Tube s5 to 8, the average value of the 2 sets (column12) showed that Tube 1 had a lower activity (0.682) than the OD in Tube 5 (0.766): therefore there is no evidence that the addition of GL-2 (in Tube 5) had any inhibitory effect on the AP enzyme.

The data also show the presence of a full complement of EG (both EGa and EGb added, total volume 1.95 mL) is not detrimental to the AP activity. By comparison of Tube 2 to Tube 1 (and the other corresponding tubes among Tubes 11 to 18) the data showed that the OD in tube 2 (which had 1.95 mL EG added) is 0.891 while the OD in Tube 1 (which had no EG) was 0.681.

The process of making spheres had an obvious negative effect on “unprotected AP”. The OD in Tubes 1, 2, 3, 4 are 0.681, 0.891, 0.420 and 0.195, respectively. Tube 3 was half-way to the making of spheres because HSA8% was added and EGa was added (but no EGb): there were no spheres present because the solution remained clear. EGa alone is not enough to cause the solution HSA molecules to de-solubilize from solution to form particles. But the AP activity as reflected by the OD is only 0.42, almost half of the OD in Tube 2 (0.891). By comparison, Tube 4 had spheres. The Whole Suspension was reacted with the phosphatase substance the same way as all the other tubes. Yet the OD was only 0.195, which is only 22% that of the OD in Tube 2.

The existence of a true “pre-mixture” of AP plus HSA (20 mg/mL in this experiment compared to 10 mg/mL in previous experiment) had a protective effect against inactivation of AP activity, as compared to a fake pre-mixture of only AP with no HSA. The OD of Tubes 11, 12, 13 and 14 were 1.120, 1.037, 1.112. 1.295, respectively. Using the OD of Tube 12 as reference, the OD of Tube 13 was higher: it means the addition of EGa to the HSA8% had no negative effect on the AP activity (which had presumably formed a conjugate with the albumin provided in the pre-mixture). In addition, the OD in Tube 14 is 1.297 which is higher than the OD in Tube 12. There was no sign of inhibition of the AP activity when a presumptive conjugate-forming mixture was present before the sphere-forming steps were instituted. The same pattern was observed in Tubes 15, 16, 17 and 18.

The data showed that the OD from Tubes 11 to 14 are consistently higher than the OD from Tubes 1 to 4. The difference is listed in the last column (OD of AP+HSA Mixture—OD of AP only). The OD from Tubes 15 to 18 is also essentially higher than the OD from Tubes 5 to 8, except for tube 16 which is almost the same as the OD from Tube 6. Part of the higher OD value in Tube 14 over Tube 4, and Tube 18 over Tube 8 can be accounted for by the protection of the AP offered by the HSA in the pre-mixture. However, it is not obvious why the OD of Tube 11 would be higher than the OD in Tube 1; or why the OD of Tube 12 would be higher than the OD of Tube 2.

### Discussion/Comments:

All the prior art disclosed herewith have shown that the formation of albumin spheres is not the result of randomly mixing certain proteins and certain chemical entities (e.g. glutaraldehyde) together. Biochemists all over the world had been adding alcohol to protein solutions and all they see are massive precipitation of the protein fractions. This is indeed how medical grades of human serum albumin are prepared in industrial scales from serum. To obtain spheres, a precise sequence of steps, including precise volumes and concentrations of various components had to be mixed together in a very short time sequence for the process to produce spheres.

### Regarding Incorporation:

It is not obvious why mixing AP directly with HSA5.5% (55 mg/mL) or HSA8% (80 mg/mL: these are the bulk material to form spheres) would not lead to the incorporation of AP into the spheres. One could form a hypothesis that AP may have repulsive forces on their molecular surfaces that would allow them to escape their capture when individual HSA molecules are coming together to form spheres. If that is the case, a mixture of AP with a lower concentration of HSA (which is 5, 10 or 20 mg/mL) would have less chance of influencing the results—but “pre-mixing” AP with a small volume of HSA in low concentrations did have an effect on AP being incorporated (Expt 4.) The fact that the concentration of HSA in the mixture is important reveals that there are molecular interactions going on which is not obvious at all, from the simple mixing of the two proteins. The data from Experiment Four showed that HSA at 5 or high (e.g. 10 mg/ml) concentration is effective while HSA at lower than 5 mg/ml is not effective. One might think that GL-2 is the agent that would link the two variety of molecules together (to form a covalent conjugate). However, this experiment showed that glutaraldehyde may not have an influence even though this experiment is not involved in measuring the degree of AP incorporation into spheres.

### Regarding Inactivation of AP:

The data here show that the presence of glutaraldehyde (i.e. GL-2) or alcohol (EGa, EGb) had minimal effect on the inactivation of AP in the final mixture. However, in the absence of protection (by the HSA in the pre-mixture) when sphere-making steps are taken, the AP will lose activity, more so when both EGa and EGb are added, than when only EGa is added. This suggests that the inactivation is due to the combined factor of at least two ingredients: (a) the presence of bulk HSA (i.e. HSA8%) which is being transformed by (b) the EG additions. The word “transformed” is used because there is no knowledge of what is actually going on at the molecular level. Soluble HSA molecules are not known to spontaneously gather together into non-soluble particles. Under inappropriate conditions, addition of alcohol to an albumin solution may form a mixture, i.e. a solution containing both albumin molecules in the soluble state and the alcohol molecules in the soluble state. Alternatively, amorphous globs of protein mass may be precipitated by the alcohol. However, we used the appropriate methods here, i.e. appropriate for the “transformation” of HSA molecules to result in spheres. The data show that “transformed HSA molecules” or “HSA molecules in the process of being transformed (by EG)” can cause the inactivation of AP (when the AP is not protected.)

To further the intrigue in this puzzle, the presence of GL-2 is not needed for the protection of the AP molecules. There may still be the formation of some kind of conjugate between the AP and the HSA molecules, but they are not likely to be covalently bonded. There is also no evidence in the literature that these two molecules will bond spontaneously, especially when their concentrations are well within physiological levels. Therefore, one would expect the AP in the pre-mixture to be diluted when the bulk HSA8% is added to the tube. So should the “protective” HSA in the pre-mixture. If these proteins in the pre-mixture are diluted into the bulk HSA8%, what then is the mechanism of protection, or the mechanism of incorporation (into the spheres)?

The data show repeatedly that the existence of a “pre-mixture” (i.e. mixed before the bulk HSA solutions are added) will offer protection to the AP against inactivation. We use only HSA solutions here of relatively low concentrations (less than 20 mg/mL). We expect a higher concentration of HSA would also be effective and that other proteins such as immunoglobulins or gelatin may also be effective.

Experiment Six: The Effect of GL-2 on the Degree of AP Incorporation into Spheres.

### Introduction

Previous experiments showed that pre-mixing AP with a small volume of HSA of at least a concentration of 5 mg/mL has an effect on protecting the AP from being inactivated during the process of making spheres, but GL-2 is not needed for the protection. Those parameters deal with the issue of protection from inactivation. This experiment evaluates the essential products from those experiments to see if the addition of GL-2 or its absence in the pre-mixture had an effect on the degree of AP incorporation into/onto spheres.

### Material and Methods:

The content of 6 tubes from Experiment Five were selected for study.

Tubes 4, 8 represented “unprotected AP” (having no HSA in the pre-mixture): Tube 4 had no GL, while Tube 8 had GL-2. From the next group of tubes, Tubes 14 and 18 represented “protected AP” i.e. there was HSA (20 mg/mL) added to the AP solution: Tube 14 had no GL, while Tube 18 had GL-2. In contrast, Tubes 24 and 28 had no AP: they were the control tubes for Tubes 4, 8 and 14, 18, respectively.

A sample from each of the 6 tubes were diluted 4× with water to a total of 1.5 mL. From this 1.5 mL of suspension, 1.0 mL was centrifuged to generate the S (Supernatant) fraction, while the remaining 0.5 mL was the WS (Whole Suspension). 0.1 mL of these fractions were set aside for protein assays, the rest for AP activity assays.

### Results:

Table 5 showed the raw OD values and the corrected OD values (after subtraction of the background values) of the WS and S fractions from the 6 tubes. From the standard curve, it was determined that the “coefficient of extinction” is 0.12256. This number is obtained from the observation that a solution of AP (12.5 ug/mL) would yield an OD of 1.532 under the condition of assay used in this experiment. Since the dilution factor is 4, the “equivalent concentration of AP” is obtained by the “corrected OD”×4/0.12256. This column is called “Equivalent Conc” because the mass of the AP protein added to 4 of these tubes was the same (Tubes 4, 8, 14, 18) but some AP might have been inactivated, making it appears that “less AP” was present in those tubes (e.g. Tubes 4 and 8).

TABLE 5
The activity of AP in various tubes from Experiment Five

| | Raw data | | | | | | | |
|---|---|---|---|---|---|---|---|---|
| | OD of | OD of | OD - Bkg | | Equivalent Conc of AP, ug/mL | | | % |
| tube | WS | Supernatant | WS | Supernatant | WS | Supernatant | Sphere | incorp |
| 4 | 0.374 | 0.282 | 0.198 | 0.132 | 6.5 | 4.3 | 2.2 | 33% |
| 8 | 0.394 | 0.293 | 0.227 | 0.138 | 7.4 | 4.5 | 2.9 | 39% |
| 14 | 1.404 | 0.843 | 1.228 | 0.693 | 40.1 | 22.6 | 17.5 | 44% |
| 18 | 1.312 | 0.803 | 1.145 | 0.648 | 37.4 | 21.1 | 16.2 | 43% |
| 24 | 0.176 | 0.15 | 0 | 0 | | | | |
| 28 | 0.167 | 0.155 | 0 | 0 | | | | |

The data showed that the activity of AP was clearly suppressed in unprotected AP solutions after the spheres were made, in that the WS fraction of Tube 4, 8 were equivalent to only 6.5 and 7.4 ug/mL of AP, while the “protected AP” solutions in Tube 14, 18 were equivalent to 40.1 and 37.4 ug/mL, respectively. Therefore, in tubes without GL-2 (Tubes 4, 14) the unprotected AP only had residual activity (6.5/40.1) i.e. 16.2% that of the protected AP. This value is very similarly to that obtained independently in Experiment Five, which was 15%. By comparison, the tubes with added GL-2 (tube 8, 18) the unprotected AP only had residual activity (7.4/37.4) i.e. 19.8% that of the protected AP. Again, this number is very close to that obtained in Experiment Five, which was 17%. These values showed that the results obtained were accurate and reliable.

The concentration of AP after spheres were made in Tubes 4, 8, 14, 18 can be calculated by the concentration of AP in the pre-mixture×volume/total volume of sphere suspension. This turns out to be 39.6 ug/mL, which is almost identical to the calculated AP concentration in the WS fraction in Tubes 14 and 18 which were converted from the measured OD and the coefficient of extinction value. Since the OD in Tubes 14 and 18 were the sum of the AP activity associated with spheres plus the activity of AP in solution, the data showed that incorporation into the interior or onto the surface of the spheres did not interfere with the activity of AP relative to the activity in solution. Also, the substrate is apparently small enough to penetrate the spheres to be acted upon by any AP incorporated in the interior of the spheres. The structure of the spheres have a “sponge-like” structure which has numerous channels connecting the interior to the surface, which provides a large “internal” surface for attachment of bioreactive agents and the passage inward of substrates and exit of the products (the yellow substance) of the AP reaction. The electron microscopy of the structure of the spheres has been published in one of Yen's prior arts.

The percent of incorporation in this experiment is 33% to 44%, regardless of whether AP is protected or not. This is a higher percentage than obtained in previous experiments probably because a slightly higher concentration of HSA (20 mg/mL) is used in this experiment. However, in the absolute sense (by weight), the amount of AP incorporated into spheres in tubes with protected AP is about 17 ug/mL of suspension. The table 6 below provided the concentration of spheres in these tubes.

The conversion from corrected OD to “protein concentration” was obtained from a standard protein solution which show that a “2 mg/mL” solution will yield an OD of 0.987. The true protein concentration of the suspension in the tube is the “converted concentration”×dilution factor.

TABLE 6
The concentration of spheres in the tubes from Experiment Five.

| | | | | | Protein concentration, | | |
|---|---|---|---|---|---|---|---|
| | | | OD - Bkg | | mg/mL | | |
| | OD of | OD of | | Super- | | Super- | |
| tube | WS | S | WS | natant | WS | natant | Sphere |
| 4 | 1.887 | 0.334 | 1.764 | 0.211 | 14.3 | 1.7 | 12.6 |
| 8 | 1.867 | 0.357 | 1.744 | 0.234 | 14.1 | 1.9 | 12.2 |
| 14 | 1.903 | 0.334 | 1.78 | 0.211 | 14.4 | 1.7 | 12.7 |
| 18 | 1.771 | 0.335 | 1.648 | 0.212 | 13.4 | 1.7 | 11.6 |
| 24 | 1.869 | 0.268 | 1.746 | 0.145 | 14.2 | 1.2 | 13.0 |
| 28 | 1.905 | 0.33 | 1.782 | 0.207 | 14.5 | 1.7 | 12.8 |

The result shows that the average concentration of spheres in all tubes is 12.5 mg/mL and the presence of AP (whether inhibited or not) has little influence on the yield of the reaction, which is 89% (=12.5/14.1).

The volume of the pellet of spheres after centrifugation from 1 mL of suspension is about 100 uL. Therefore, the concentration of AP in the spheres in tube 14 is about 17.5 ug/0.1 mL, which is 175 ug per mL of sphere volume. This is more than 4 times that of the “overall AP concentration in the suspension” which is 40.1 ug/mL of suspension.

Experiment Seven: Incorporation of a Bioreactive Agent that is Resistant to Inactivation or Only Mildly Inactivated During Formation of Spheres, e.g. Urokinase.

### Introduction

Many bioreactive agents are protein enzymes—their major function is to catalyze the break-up or joining-together of their substrates. There is no particular biochemical or structural reason to suspect that they have different categories in terms of their being able to remain active duration the process of, or after being encapsulated into albumin spheres. However, the data in the previous experiments did show that one bioreactive agent, e.g. alkaline phosphatase will become at least partially inactivated (the mechanism unknown) during the process of the formation of spheres from soluble albumin molecules. That observation is surprising because the “bulk material” to make the spheres is HSA (e.g. starting at 8% w/v) but the material needed to protect alkaline phosphatase enzymes from inactivation (the “protective material”) is also HSA (e.g. 2%).

Experiment seven is an attempt to evaluate if another bioreactive agent, e.g. urokinase (UK), is equally susceptible to inactivation during the process of making spheres, or whether UK does not need special steps of protection to remain enzymatically active during and after encapsulation into albumin spheres. There are many terms to describe the robustness of an enzyme during and after encapsulation into albumin spheres, here we use the term “resistant to inactivation.”

### Materials and Methods:

The Table 7 below listed the steps involved in the encapsulation of soluble UK molecules into albumin spheres. UK is purchased from ABCAM, so is the Urokinase type plasminogen activator Human Chromogenic Activity Assay Kit ab108915.

Essentially, Tube F is the “unprotected” procedure, where a solution of UK is later mixed with the bulk material designed to form albumin spheres.

Tube G is the “protected” procedure where a solution of UK is first mixed with an equal volume of HSA2% for at least 30 minutes to form a potential protective “conjugate.” We use the term “conjugate” in a generic sense because the HSA2% in the case of alkaline phosphatase must have interacted in some biochemical way to offer protection of the alkaline phosphatase against inactivation by the “transformed HSA molecules” (albumin molecules which are on their way to sticking together to form into spheres.)

Tube H is the tube where spheres are formed without the presence of UK, to control for OD disturbances caused by the spheres or left-over material. It has been noted that spheres can cause a false increase in the optical density values; therefore, the spheres will be removed before the OD of any reactions are read.

Tube J is the tube where the UK solution is diluted with water (in replacement of the bulk material to make spheres.) This provided the OD value expected from an uninhibited UK solution.

The UK reaction is based on the kit bought from AB Cam ( ). It is a 2-step reaction: first, the UK in the test material will convert the plasminogen (provided in the kit) to plasmin. The plasmin will then convert a “plasmin substrate” into a product which is yellow and can be read at wavelength 405 nM.

TABLE 7
Key steps in the incorporation of urokinase (UK) into albumin spheres, and controls

| | | | Bulk Material | | | | |
|---|---|---|---|---|---|---|---|
| | | | added to | | | Any | |
| | Pre-mixture | | make spheres | | Fibrinogen | aggregates | Total vol of |
| | UK | | HSA6%, | Spheres | solution | in the FAS | suspension or |
| Tube | solution | HSA2% | GL-1, EG | formed | added | suspension? | solution,mL |
| F | 25 uL | no | yes | yes | yes | no | 680 uL |
| G | 25 uL | 25 uL | yes | yes | yes | no | 680 uL |
| H | no | 25 uL | yes | yes | yes | no | 680 uL |
| J | 25 uL | no | no | no | no | n/a | 680 uL |

Essentially, the stock UK solution (1.57 mg/mL) was diluted tenfold to 157 ug/ml with water and used (25 uL) to mix with water (Tube F) or HSA2% (Tube G). Where “no” is indicated in Table 7, an equal volume of water is used to substitute for the other material, e.g. in making spheres. Due to the salt content in the UK solution, the spheres made with UK solutions are larger than expected from spheres made from the HSA6% without the UK solution added. To avoid the formation of aggregates, the volume of EG used is decreased. Here, the volume of HSA6%, GL-1, EGa and EGb are 100, 50, 110, 200 uL, respectively. Again, the addition of GL-1 (0.15 mg/mL) marks the beginning (time zero) of making spheres. EGa and EGb are added at time 60 and 150 seconds, respectively. A sample of the spheres were examined under the microscope. The spheres in Tubes F, G were about one micron in diameter while the spheres in Tube H is slightly smaller. None had aggregates or spheres larger than 5 microns. After 3 to 4 hours of stabilization of the spheres against resolubilization, a solution of fibrinogen (concentration at 1.4 mg/mL in water containing also sodium tetradecyl sulfate at 1 mg/mL) was added 1 vol per 3 vol of “sphere-suspension-before-fibrinogen addition”, i.e. 170 uL of fibrinogen solution added to 510 uL of the sphere suspensions.

To separate the fractions, 500 uL of the suspensions Tubes F, G, H are centrifuged for 5 minutes to allow the spheres to form a pellet. The remaining uncentrifuged portion of 180 uL is called the WS fraction (Whole Suspension.) After centrifugation, about 300 uL of the top part of the supernatant (Fraction S) is carefully removed without disturbing the pellet. Then as much as possible of the fluid above the pellet is removed and discarded. The volume of the pellet is estimated to be about 50 uL. Therefore, 450 uL of water is used to resuspend the pellet into the Sphere fraction (500 uL total volume.) In previous experiments, the activity of the Sphere fraction is obtained by the “Activity in WS” minus “Activity in S fraction.” Here we recognize that the Sphere fraction may not have all the spheres in the original suspension because some might have been removed during the step to remove all the supernatant fluids. But the direct measure of the Sphere Fraction will validate that the spheres do have enzyme activity associated with it.

Ideally, the data may be more precise if there is a step available to stop the UK reaction so that the OD is stable while the spheres are removed. But since the UK assay does not provide a step to stop the reaction, we use “dilution with water” as a method to slow the speed of the reaction as well as a mean to remove any spheres in the reaction mixture. To obtain multiple time points, each tube will have the following ingredients in the “reaction tube”: Diluent (70 uL), Plasminogen (20 uL), Plasmin substrate (30 uL). To start the reaction, 10 uL of the content of the various tubes are added to the mixtures in the reaction tubes. To obtain the “zero time point” 30 uL of the reaction mixture is immediately removed after the 10 uL of sample was added to the mixture, and mixed with 700 uL of water in a 1.5 mL ultracentrifuge tube. The 730 uL of mixture is centrifuged for 5 minutes to remove any spheres present. Then OD of the fluid is read immediately in a curvette in a standard spectrophotometer. Meanwhile the reaction in the reaction-tube is still going on (with or without spheres, with or without UK) and at various later time points, a similar step of taking out 30 uL of the reaction mixture will be performed (and diluted into 700 uL of water and centrifuged to remove spheres).

The content of Tube J provided the expected OD for the concentration of UK used as in a standard curve. The concentration of the UK solution (25 uL) added to the reaction mixture is 157 ug/mL. Since the subsequent steps of reaction and OD reading are the same for all tubes, we will refer the activity of Tube F, G, H by comparison of their respective OD to the OD (in the 730 diluted solution) obtained from tube J.

### Results:

Table 8 show the corrected OD of the various tubes (corrected by subtraction of the background OD brought in by the phosphatase substrate, as reflected in tube L, average value is about 0.09.)

Therefore, after correction the value at various times in Tube L is zero.

It is observed that the maximum OD obtained from this assay method is about 0.2. This is not the upper limit of the spectrophotometer. Rather it is the upper limit of the substrate:

being used up by the highly active UK preparations by or before 45 minutes. Therefore, the slope is the OD difference between the OD after 15 minutes of incubation (reaction time) minus OD at time zero, even though the reaction is near “exhaustion” (substrate being completely used up.)

We observe that the resuspended Sphere Fraction consistently have activity higher than that of [WS-S] fractions.

TABLE 8
Activity of various preparations

| | Obtain from | | | | | | | slope |
|---|---|---|---|---|---|---|---|---|
| Tube | Reaction Tube | Fraction | 0 MIN | 15 MIN | 30 MIN | 45 MIN | Slope | (WS − S) |
| 1 | F | WS | 0 | 0.059 | 0.09 | 0.181 | 0.059 | |
| 2 | | S | −0.005 | 0.028 | 0.037 | 0.083 | 0.033 | |
| 3 | | SPHERE | 0.032 | 0.154 | 0.205 | 0.189 | 0.122 | 0.026 |
| 4 | G | WS | 0.012 | 0.074 | 0.116 | 0.178 | 0.062 | |
| 5 | | S | −0.014 | 0.015 | 0.034 | 0.079 | 0.029 | |
| 6 | | SPHERE | −0.014 | 0.174 | 0.191 | 0.196 | 0.188 | 0.033 |
| 7 | H | WS | −0.005 | 0.001 | 0.001 | −0.004 | 0.006 | |
| 8 | | S | −0.009 | 0.004 | −0.006 | −0.002 | 0.013 | |
| 9 | | SPHERE | −0.008 | 0.001 | −0.003 | −0.009 | 0.009 | −0.007 |
| 10 | J | UK IN | 0.003 | 0.072 | 0.125 | 0.201 | 0.069 | |
| | | SOLUTION | | | | | | |
| 11 | L | N/A | 0 | 0 | 0 | 0 | | |

The data show that the slope of Tubes F and G from the “reconstituted sphere fraction” is 0.122 and 0.188, respectively. The slope of Tube H (where no UK is added), as expected, is practically 0.

It is currently not understood why the Sphere Fraction has so much higher activity than the difference between WS and S (i.e. “slope of WS-S’, last column). It can be argued that the WS and S fractions still contain a high concentration of ethanol (about 40%) while the reconstituted Sphere fraction has practically no alcohol (being reconstituted with water of the same volume.) However, the control slope of tube 10 from J (which has no alcohol) shows a value of 0.072 at 15 minutes, which is very close to the value of 0.074 as shown in Tube 4 from the WS fraction.

Therefore, the hypothesis that alcohol might have suppressed the activity of WS in Tubes 4 and 1 is not correct. In other words, the activity of UK within the spheres (plus the left over in the supernatant fraction) is as active as the UK enzymes in the water-only J preparation.

The concentration of spheres in the WS Fractions is the same as the respective reconstituted Sphere Fraction. Therefore, the higher activity of the Spheres Fractions compared to [WS-S] is a surprise. It is not expected nor obvious from all previous experiences or published literature. Nobody to our knowledge as measured the activity of encapsulated enzymes and show that it is higher than the same mass of enzyme in solution. In our spheres, the activity of the Sphere Fraction is substantially higher than expected, being 0.188/0.069 (Tube 6 compared to Tube 10, which is 0.072-0.03) which is 272% that of the equivalent enzyme solution.

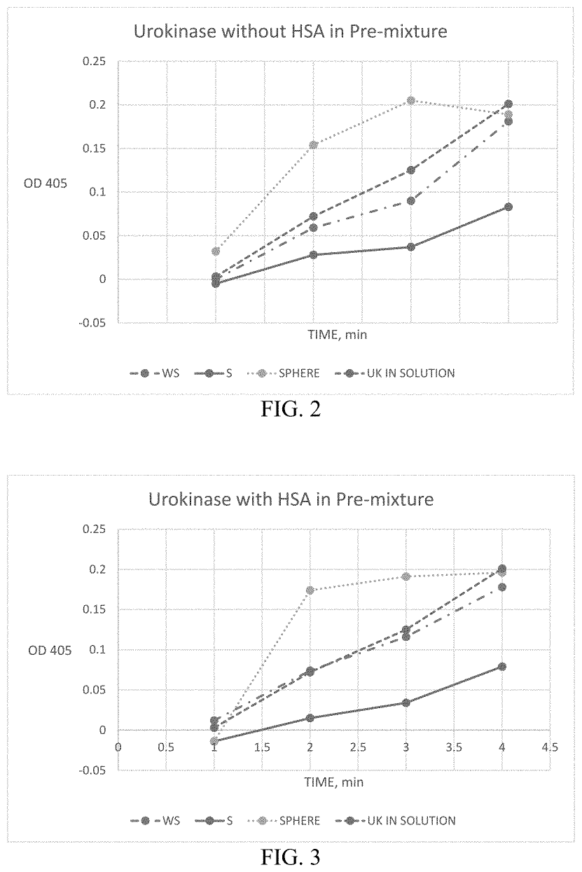

FIGS. 2 and 3 show the slopes of the various reaction mixtures (after dilution into water to have a volume of 730 uL which allows the spectrophotometer to read the colored solution.) The “unprotected” approach is presented in FIG. 2 where UK is mixed with water, before the bulk material is added to make spheres and illustrates the activity of spheres made without HSA in the pre-mixture (unprotected UK). FIG. 3 showed the kinetics when UK is mixed with HSA2% in the pre-mixture and illustrates the activity of spheres made with HSA2% in the pre-mixture (protected UK).

### Discussion

Both Table 8 and the two graphs shown in FIGS. 2 and 3 show that the “protected UK” has a higher activity than the “unprotected”—the slopes of the reconstituted Sphere Fraction being 0.188 vs 0.122, respectively. This is a difference of 0.066/0.122, or 54%. However, if we use the slope of the WS fraction, the values are 0.062 and 0.059, respectively. This is a difference of only 0.003/0.059, which is 5%. Since we do not understand the “over-activity” of the reconstituted sphere fraction, we will rely on the activity of WS, especially by comparison to the control (UK in solution). We will call a bioreactive molecule susceptible to inactivation if the activity in the unprotected approach is 25% lower than the activity in the protected spheres, or the same mass of enzyme in solution. By this arbitrary definition, UK belongs to the category that needs no protection.

However, we prefer to offer protection to the manufacturing of UK spheres, because the Sphere Fraction (the active drug substance in any future drug product) is noticeably in activity than the unprotected UK species within the spheres. Note: the term “protected” vs “unprotected” only refer to the situation of the enzyme in the pre-mixture against the potential harm from the process in the making of the spheres. The UK enzymes are protected from dilution or potential inactivation from inhibitors, by virtue of their association with the spheres, in both cases.

### Experiment Eight: Functionality of Both Protected UK Spheres and Unprotected UK Spheres.

### Introduction

It is important that (a) the spheres can be targeted to clots in vitro and in vivo, (b) the molecules responsible for dissolving the clot can actually dissolve the clot.

The preparations F, G, H of the previous experiment all have fibrinogen added and the data show that this step caused no harm to the preparation (no clumping or formation of aggregates as observed under the microscope.) The presence of fibrinogen on the sphere surface will allow attachment to the clot during clot-formation or long after that (because fibrin molecules, formed by action of thrombin on the fibrinogen molecules on the surface of the spheres will be converted to fibrin, which will attach to any fibrin, including old fibrin stacks in the old clot.)

This experiment is designed to see if soluble plasminogen molecules can enter the spheres and be converted into plasmin and that the plasmin can diffuse out of the spheres to dissolve the fibrin clot structure outside the spheres.

### Material and Methods:

Demonstration that anti-fibrinogen IgG can attach to the spheres in Preparation F, G, and H (which had fibrinogen added to the spheres) but not spheres without added fibrinogen.

Two new sphere preparation were made similar to preparation F above as shown in Table 9: K will be the same as the conventional Fibrinogen-coated albumin spheres (FAS) and M will be the Control spheres (CS) without fibrinogen.

TABLE 9

| | | | Bulk Material | | | | |
|---|---|---|---|---|---|---|---|
| | | | added to | | | Any | |
| | Pre-mixture | | make spheres | | Fibrinogen | aggregates | Total vol of |
| | UK | | HSA6%, | Spheres | solution | in the FAS | suspension or |
| Tube | solution | HSA2% | GL-1, EG | formed | added | suspension? | solution,mL |
| K | no | no | yes | yes | yes | no | 680 uL |
| M | no | no | yes | yes | no | no | 680 uL |

Pilot experiments have shown that CS have a high capacity to absorb non-specific IgG. Therefore to an aliquot of preparation F, G, K, M, non-specific rabbit IgG was added to coat the non-specific binding sites for one hour before a dilute solution of Goat anti-human-fibrinogen Antibody (linked to peroxidase, bought from Rockland, Lot #32849) was added to attach specifically to fibrinogen that have attached previously during the sphere manufacturing process. Thereafter, the sphere preparation was centrifuged to obtain the sphere pellet. The supernatant fraction containing surplus non-specific rabbit IgG and extra anti-human-fibrinogen antibody was discarded. The pellet was resuspended in water. Due to the abundance of negative charges (even after attachment of the antibody) on the surface of the spheres, the spheres will repulse each other and auto-resuspend when left undisturbed over night in water. The reconstituted sphere fraction was added to a OPD solution to obtain the activity of the peroxidase associated with the antibody. After about 30 minutes, the reaction was stopped with hydrochloric acid. The color solution is then centrifuged again to remove the spheres and the OD of the reacted solution is read at 492 nM.

Demonstration that plasminogen can enter the spheres and plasmin can exit the spheres to dissolve a fibrin clot surrounding the spheres.

Fibrinogen was purchased from a commercial source (Desert Biologics, AZ) and dissolved in normal saline to form a solution of 10 mg/mL. The sphere suspensions of Tubes F, G, H (500 uL) were centrifuged to obtain the sphere fraction (volume about 50 uL, which was resuspended with 450 uL of water.) Two solutions were prepared for comparison: Preparation J is a UK solution (25 uL at 0.157 mg/ml diluted to a total volume of 6.8 mL (as in Table 7). Preparation L is water as a control.

To form a clot, the following steps are performed: 500 uL of fibrinogen solution (10 mg/mL), 100 uL of UK sample (preparations F, G, H, J, L) and 50 uL of the plasmin-substrate (obtained from the UK kit from ABCAM) were mixed well in a 1.5 mL polyethylene centrifuge tube. The plasmin-substrate is not needed for the reaction, except that it will provide a yellow color when plasmin is formed, by the conversion of plasminogen (which will be converted to plasmin.) This will allow an estimation of whether the fibrin clot (to be formed) is in the process of being (slowly?) digested. This mixture contained no added plasminogen. Finally, 50 uL of a thrombin solution is added (1 Ku dissolved in 2 mL of water). After mixing the contents, the centrifuge tube is inverted to allow the formation of a button-shaped clot near the flap-cap of the centrifuge tube.

Within 5 minutes of the addition of the thrombin solution, a solid plug of a clot is seen in all tubes. However, within 10 minutes, the clot in tube J turned yellow. This means there is plasminogen present in the mixture, most likely from the contaminating amounts of plasminogen common in commercial preparations of fibrinogen powder. The clots are left over night to solidify. The plan is to add plasminogen solution to the clots after 8 hours to see if the clots can dissolve from the plasmin converted from the plasminogen entering the spheres, where they are converted to plasmin.

### Results:

The OD of the various preparation after interaction with OPD substrate showed that preparations F, G, H and K all have anti-fibrinogen antibody attachment while preparation M had minimal attachment of the anti-fibrinogen antibody. The data showed that encapsulation of UK into the spheres (or on the spheres surface) had no adverse effect on the subsequent attachment of fibrinogen. This is true whether the UK is encapsulated in the unprotected approach (Tube F) or protected approach (Tube G.) The content of Tube H is essentially the same as Tube K, both having no UK, but have fibrinogen coating.

Fibrinogen is used as the targeting molecule, but it can be appreciated that other molecules will be also effective depending on what target we want the spheres to attach to. For example, we can attach a specific antibody to the surface of our spheres (which may carry other bioreactive molecules) and use such IgG to target other cell or tissue carrying the appropriate antigen.

The data show that overnight, the amount the plasminogen in the fibrinogen clot is enough for conversion into plasmin that the clots in the tubes with UK (whether encapsulated or in solution) will be able to dissolve the clot. The following Table 10 show the results.

TABLE 10

| | | Solid clot formed by | |
|---|---|---|---|
| | | fibrinogen in mixture | |
| Prepa- | | after addition of | |
| ration | UK present? | thrombin? | Clot dissolved |
| F | Yes, | Yes | Yes, yellow solution |
| | encapsulated | | left in tube |
| G | Yes, | Yes | Yes, yellow solution |
| | encapsulated | | left in tube |
| H | no | Yes | Clot stays |
| J | Yes, in | Yes | Yes, yellow solution |
| | soluble form | | left in tube |
| L | No, only water | yes | Clot stays |

### Discussion

One can readily see the difference between a thrombolytic enzyme in solution vs the same molecules encapsulated in spheres as shown in Table 11.

TABLE 11

| | | Enzymes encapsulated in |
|---|---|---|
| Features | Enzymes in solution | spheres |
| High | May be difficult to | Can achieve high |
| concentrations | dissolve, nor stable in | concentrations within the |
| in the product | long durations | spheres, mainly because |
| formulation | | the spheres have very |
| | | small volumes. One sphere |
| | | with 100 nm diameter has a |
| | | volume of only 5.3 × 10 |
| | | [−16] cubic cm. |
| Dilution after | The soluble enzymes will | The enzyme within the |
| injection | distribute in the blood | sphere stays the same, |
| into a patient | volume (and total body | regardless of disperse the |
| | fluid if it can pass the | spheres are after |
| | blood vessel barrier) | administration |
| | | intravenously |
| Degradation by | The soluble enzyme or | The spheres will travel |
| the liver | bioreactive molecules | along the endothelium and |
| | will be recirculated to | not quickly circulated to |
| | the liver for degradation. | the liver for degradation. |
| | Therefore the total mass | One dose of the typical |
| | of the remaining enzyme | sphere can last at least 5 |
| | will quickly decrease | days in vivo and exert their |
| | | beneficial effects. Spheres |
| | | trapped in clots will not be |
| | | circulated to the liver until |
| | | the clot is dissolved. |
| Effect on a | Only a small fraction of | The fibrinogen on the |
| vaso-occlusive | the entire injected dose | surface of the sphere will |
| clot | can pass over the clot, | allow capture of the |
| | the rest of the dose is | fibrinogen-coated loaded |
| | somewhere else | thrombolytic spheres by |
| | | the clot whether the clot is |
| | | actively being formed or an |
| | | old clot, because fibrin |
| | | molecules tend to stack up |
| | | on other fibrin molecules |
| Reactivity with | The plasminogen will be | The data here show that |
| urokinase | present in soluble form in | soluble plasminogen can |
| substrates, e.g. | the plasma. The | easily pass into the spheres. |
| plasminogen | concentration of | The UK inside the spheres |
| | plasminogen may be | can process the |
| | constant under a given | plasminogen. Only very |
| | physiological condition, | small quantities of |
| | but the concentration of | plasminogen are needed, as |
| | urokinase, for example | the amount of plasminogen |
| | may be very dilute and | in the fibrinogen solution |
| | not optimal for process of | used to make the fibrin clot |
| | the plasminogen | is only at “contamination |
| | | levels.” The product |
| | | plasmin can easily diffuse |
| | | out of the spheres. This is |
| | | shown by the fact that the |
| | | fibrin clot is readily |
| | | digested without the |
| | | addition of extra |
| | | plasminogen (except the |
| | | small amount already |
| | | present in the commercial |
| | | fibrinogen preparation.) |
| Effect on clot | The plasmin converted | The spheres are attached to |
| | from plasminogen by | the clots already. Any |
| | urokinase may not be | plasmin converted by the |
| | high enough or fast | high concentration of UK |
| | enough (before the | inside the spheres will |
| | plasmin is washed away | quickly diffuse out of the |
| | or attaching downstream) | spheres. Since the spheres |
| | to quickly dissolve the | are already attached to the |
| | clot. | clot, the plasmin does not |
| | | have to “find the clot.” |
| | | Therefore, the plasmin will |
| | | not suffer dilution by the |
| | | blood stream passing by |
| | | but can work once they |
| | | leave the sphere. |
| Re-use of the | The concentration of the | The concentration of the |
| enzyme | enzyme will decrease | spheres cannot be easily |
| | with time | measured from blood |
| | | sample because the |
| | | majority of spheres stay |
| | | near the endothelium or |
| | | within the capillaries. |
| | | Spheres trapped inside a |
| | | clot in vivo will stay there |
| | | until the clot is dissolved, |
| | | then the spheres will travel |
| | | harmlessly until they meet |
| | | another clot where they |
| | | will start working again. |
| | | The spheres can be re- |
| | | cycled. |
| Enzyme | The activity of the | The data show that UK has |
| Activity | dissolved enzyme | a much higher activity |
| | follows it concentration | when associated with |
| | in vitro or in vivo | spheres, than predicted |
| | | from the activity of the |
| | | same mass of enzyme in |
| | | solution. Therefore, a |
| | | small quantity used to |
| | | make spheres will be |
| | | economically the same as a |
| | | larger quantity to be used |
| | | in solution |
| Side Effects | Thrombolytic solutions at | Thrombolytic spheres will |
| | high enough | always have the same |
| | concentrations in | concentration within a |
| | locations not having clots | sphere. They will |
| | may cause bleeding. If | concentrate only when they |
| | the bioreactive agent can | meet a clot. The |
| | provoke the formation of | antigenicity of any |
| | antibodies, they will. | bioreactive agent will be |
| | | decreased because they are |
| | | not exposed to the antibody |
| | | formation mechanism - the |
| | | agent being hidden within |
| | | the spheres |

According to one aspect, the present technology can include a thrombolytic microsphere configured to dissolve vaso-occlusive clots. The microsphere can comprise a submicron albumin sphere, and an enzymatically active bioreactive agent encapsulated in the albumin sphere. The albumin sphere can be configured to dissolve vaso-occlusive clots in vivo.

According to another aspect, the present technology can include a composition for dissolving vaso-occlusive clots in a subject in need thereof. The composition can comprise a therapeutically effective amount of an albumin nanoparticle suspension containing submicron albumin spheres, and an enzymatically active bioreactive agent encapsulated in the albumin spheres. The albumin spheres can be configured to dissolve vaso-occlusive clots in vivo.

According to yet another aspect, the present technology can include a method of dissolving vaso-occlusive clots in a subject in need thereof. The method can include the steps of administering a therapeutically effective amount of an albumin nanoparticle suspension containing submicron albumin spheres, and an enzymatically active bioreactive agent encapsulated in the albumin spheres to the subject. The albumin spheres can be configured to dissolve vaso-occlusive clots in vivo.

In some or all embodiments of the present technology, the enzymatically active bioreactive agent can be urokinase.

In some or all embodiments of the present technology, the submicron albumin sphere can be a plurality of submicron albumin spheres contained in an albumin nanoparticle suspension.

In some or all embodiments of the present technology, the albumin nanoparticle suspension can be prepared by combining a pre-mixture including urokinase and a first human serum albumin with a bulk material including a second human serum albumin, glutaraldehyde and ethyl alcohol.

In some or all embodiments of the present technology, the albumin nanoparticle suspension can further include fibrinogen.

In some or all embodiments of the present technology, the fibrinogen can be a solution of fibrinogen and sodium tetradecyl sulfate.

In some or all embodiments of the present technology, the enzymatically active bioreactive agent can be urokinase.

Some or all embodiments of the present technology can include the step of preparing the albumin nanoparticle suspension by combining a pre-mixture including the urokinase and a first human serum albumin with a bulk material including a second human serum albumin, glutaraldehyde and ethyl alcohol.

In some or all embodiments of the present technology, the ethyl alcohol can be added to the bulk material as a first ethyl alcohol portion and a second ethyl alcohol portion different to that of the first ethyl alcohol portion.

In some or all embodiments of the present technology, the first human serum albumin can be at 2%, and the second human serum albumin can be at 6%.

In some or all embodiments of the present technology, the second human serum albumin can be at a volume of 100 uL, the glutaraldehyde can be at a volume of 50 uL, the first ethyl alcohol portion can be at a volume of 110 uL, and the second ethyl alcohol portion can be at a volume of 200 uL.

Some or all embodiments of the present technology can include the step of adding a solution of fibrinogen and sodium tetradecyl sulfate the albumin nanoparticle suspension after combining the pre-mixture and the bulk material.

In some or all embodiments of the present technology, the administering of the albumin nanoparticle suspension can be intravenously.

What is claimed is as follows:

1. A thrombolytic microsphere to dissolve vaso-occlusive clots comprising a submicron albumin sphere, and an enzymatically active bioreactive agent encapsulated in the albumin sphere, wherein the albumin sphere being configured to dissolve vaso-occlusive clots in vivo.

2. The thrombolytic microsphere according to claim 1, herein the enzymatically active bioreactive agent is urokinase.

3. The thrombolytic microsphere according to claim 2, wherein the submicron albumin sphere is a plurality of submicron albumin spheres contained in an albumin nanoparticle suspension.

4. The thrombolytic microsphere according to claim 3, wherein the albumin nanoparticle suspension is prepared by combining a pre-mixture including urokinase and a first human serum albumin with a bulk material including a second human serum albumin, glutaraldehyde and ethyl alcohol.

5. The thrombolytic microsphere according to claim 4, wherein the albumin nanoparticle suspension further includes fibrinogen.

6. The thrombolytic microsphere according to claim 5, wherein the fibrinogen is a solution of fibrinogen and sodium tetradecyl sulfate.

7. A composition for dissolving vaso-occlusive clots in a subject in need thereof, the composition comprising a therapeutically effective amount of an albumin nanoparticle suspension containing submicron albumin spheres, and an enzymatically active bioreactive agent encapsulated in the albumin spheres, wherein the albumin spheres being configured to dissolve vaso-occlusive clots in vivo.

8. The composition according to claim 7, herein the enzymatically active bioreactive agent is urokinase.

9. The composition according to claim 8, wherein the albumin nanoparticle suspension is prepared by combining a pre-mixture including the urokinase and a first human serum albumin with a bulk material including a second human serum albumin, glutaraldehyde and ethyl alcohol.

10. The composition according to claim 9, wherein the albumin nanoparticle suspension further includes fibrinogen.

11. The composition according to claim 10, wherein the fibrinogen is a solution of fibrinogen and sodium tetradecyl sulfate.

12. A method of dissolving vaso-occlusive clots in a subject in need thereof, the method comprising the steps of administering a therapeutically effective amount of an albumin nanoparticle suspension containing submicron albumin spheres, and an enzymatically active bioreactive agent encapsulated in the albumin spheres to the subject, wherein the albumin spheres being configured to dissolve vaso-occlusive clots in vivo.

13. The method according to claim 12, herein the enzymatically active bioreactive agent is urokinase.

14. The method according to claim 13, wherein the albumin nanoparticle suspension further includes fibrinogen.

15. The method according to claim 14 further comprising the step of preparing the albumin nanoparticle suspension by combining a pre-mixture including the urokinase and a first human serum albumin with a bulk material including a second human serum albumin, glutaraldehyde and ethyl alcohol.

16. The method according to claim 15, wherein the ethyl alcohol is added to the bulk material as a first ethyl alcohol portion and a second ethyl alcohol portion different to that of the first ethyl alcohol portion.

17. The method according to claim 16, wherein the first human serum albumin is at 2%, and the second human serum albumin is at 6%.

18. The method according to claim 17, wherein the second human serum albumin is at a volume of 100 uL, the glutaraldehyde is at a volume of 50 uL, the first ethyl alcohol portion is at a volume of 110 uL, and the second ethyl alcohol portion is at a volume of 200 uL.

19. The method according to claim 16 further comprising the step of adding a solution of fibrinogen and sodium tetradecyl sulfate the albumin nanoparticle suspension after combining the pre-mixture and the bulk material.

20. The method according to claim 11, wherein the administering of the albumin nanoparticle suspension is intravenously.
